(19) United States
(12) Patent Application Publication
(10) Pub. No.: US 2016/0031845 A1
(43) Pub. Date: Feb. 4, 2016

(54) IN-SITU GENERATION OF RUTHENIUM CATALYSTS FOR OLEFIN METATHESIS

(71) Applicant: EVONIK INDUSTRIES AG, Essen (DE)

(72) Inventors: Renat Kadyrov, Frankfurt (DE); Christa Dumrath, Schwerin (DE); Andreas Dumrath, Schwerin (DE); Helfried Neumann, Rostock (DE); Matthias Beller, Nienhagen (DE)

(21) Appl. No.: 14/774,862

(22) PCT Filed: Feb. 13, 2014
(86) PCT No.: PCT/EP2014/052765

§ 371 Date: Sep. 11, 2015

(30) Foreign Application Priority Data
Mar. 13, 2013 (EP) 13158985.5

(51) Int. Cl. C07D 313/08; C07C 41/18; C07C 67/333; C07D 207/48; B01J 31/24; B01J 31/22

(52) U.S. Cl. CPC C07D 313/08; B01J 31/24; B01J 31/2295; B01J 31/2265; C07C 67/333; C07D 207/48; C07C 41/18; B01J 2531/821; C07C 2101/10; C07C 2101/16

(57) ABSTRACT

The present invention relates to a process for preparing olefins by means of metathesis, which comprises the following steps
a. provision of an olefin reaction mixture containing at least one olefin,
b. addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c. addition of a Lewis acid and/or an anionic, noncoordinating salt,
d. reaction at temperatures in the range from 30° C. to 140° C.,
where no addition of an alkyne or alkynol is carried out.

The invention further relates to the use of this process in metathesis reactions.

The present invention relates to a process for preparing olefins by means of metathesis in the presence of Ru catalyst complexes generated “in situ”.

Olefin metathesis has become established as an effective carbon-carbon coupling reaction in recent decades and is widely employed in organic synthesis and polymer science. Ruthenium-carbene catalysts, in particular, and their derivatives have firmly anchored olefin metathesis as a versatile and reliable synthetic method in demanding organic synthesis. The extremely wide range of substrates and also great tolerance towards a wide variety of functional groups make olefin metathesis a useful, fast and efficient technique for synthesizing molecules which can otherwise be obtained only with difficulty via traditional organic synthesis. Many different transition metal complexes can serve as catalysts in olefin metathesis. In particular, specific ruthenium- and osmium-carbene compounds are effective catalysts in olefin metathesis reactions such as cross-metathesis (CM), ring-closing metathesis (RCM), ring-opening metathesis (ROM), ring-opening metathesis polymerization (ROMP) or acyclic diene metathesis (ADMET).

Particularly active olefin metathesis catalysts usually bear both phosphane ligands and nucleophilic heterocyclic carbene ligands (NHC ligands) and additionally have a metal-carbene structure. A prominent example of this type of olefin metathesis catalysts are the sometimes commercially available Ru complexes having the general structure RuX₂(═CR₂)LL′, where X is an anionic ligand, R is selected from the group consisting of hydrogen, (C₆-C₁₄)-aryl and (C₃-C₁₄)-heteroaryl radicals and L and L′ are each an uncharged electron donor ligand, where L is an N-heterocyclic carbene and L′ is a phosphane.

The synthesis of these ruthenium-carbene complexes is comparatively complicated, raw materials-intensive and expensive. Typical synthetic methods consist of a plurality of stages, with complicated process conditions under an inert gas atmosphere, starting materials which are not readily available or the use of reactants which are problematic in terms of safety sometimes being required. In particular, the use of carbene precursors, e.g. disubstituted cyclopropenes (WO 93/20111), diazoalkanes (WO 97/06185) or acetylenes (DE 19854869), represent a considerable safety risk on an industrial scale and should consequently be avoided. Metal-organic starting materials such as RuCl₂(PPh₃)₃ (Hill et al., Dalton 1999, 285-291) or RuHCl (PPh₃)₃ (Hoffmann et al., Journal of Organometallic Chemistry 2002, 641, 220-226) are prepared from RuCl₃ using a large excess of triphenylphosphane (PPh₃), with these PPh₃ ligands being lost as a result of a ligand exchange reaction in the subsequent catalyst synthesis. The most recent synthesis improvements can partly compensate for this disadvantage by direct reaction of tricyclohexylphosphane with ruthenium chloride hydrate or Ru(cod)Cl₂ to form the (PCy₃)₂RuCl₂-carbene complexes (WO 2009/124977). However, large amounts of PCy₃ or PCy₃ solution continue to be required. In addition, one of these PCy₃ ligands is replaced by an uncharged electron donor ligand in the synthesis of the 2^{nd} generation of ruthenium-carbene complexes, as a result of which PCy₃ is once again lost from the complex. Inexpensive alternatives to ruthenium-phosphane complexes are neutral or anionic ruthenium-aryl complexes having the general structure [RuX(═C═[C]ₙ═CR₂)L¹L²]Y (EP0921129A1). X and R are defined as described above, the ligands Y are anionic, weakly coordinating ligands, the ligands L¹ are phosphanes, phosphites, phosphonites, phosphinites, arsanes or stibines, L² is benzene or a substituted benzene derivative such as p-cymene. Another disadvantage is that the structural unit Ru═C=[C]ₙ═CR₂ is relevant for the activity of the ruthenium-aryl complexes, and this in turn requires the use of hazardous, difficult-to-obtain or very sensitive carbene precursors.

Many examples of catalytic systems generated “in situ” from available ruthenium precursors have already been described as an alternative approach. However, to produce the active catalyst, use is made of either
1) a photochemical activation (A. Hafner, A. Mühlebach, P. A. van der Schaaf, Angew. Chem. 1997, 109, 2213-2216; Angew. Chem. Int. Ed., 1997, 36, 2213-2216., L. Delaude, A. Demonceau and A. F. Noels, Chem. Commun., 2001, 986-987., A. Fürstner, and L. Ackermann, Chem. Commun., 1999, 95-96, L. Jafarpour, J. Huang, E. D. Stevens, and S. P. Nolan Organometallics 1999, 18, 3760-3763)
2) an activation via carbene precursors such as trimethylsilyldiazomethane (a) A. W. Stumpf, E. Saive, A. Demonceau and A. F. Noels, Chem. Commun., 1995, 1127; b) A. Demonceau, A. W. Stumpf, E. Saive and A. F. Noels, Macromolecules, 1997, 30, 2127), or
3) activation using alkynes (Y. Miyaki, T. Onishi, S. Ogoshi, H. Kurosawa, J. Organometallic Chem. 2000, 616, 135-139., J. Louie, R. H. Grubbs, Angew. Chem. Int. Ed. 2001, 40, 247-9., D. Semeril, C. Bruneau, P. H. Dixneuf, Helv. Chim. Cata 2001, 84, 3335-3341; D. Semeril, C. Bruneau, P. H. Dixneuf Adv. Synth. Catal. 2002, 344, 585-595).

It has also been reported that homobimetallic Ru—NHC complexes can initiate ring-opening metathesis without photochemical or chemical activation (X. Sauvage, Y. Borguet, A. F. Noels, L. Delaude, A. Demonceau, Adv. Synth. Catal. 2007, 349, 255-265). This gives a mixture of cycloisomerization and RCM products when α,ω-dienes are reacted in the presence of these complexes.

It is therefore an object of the present invention to provide a process for preparing olefins by means of metathesis, in which the catalyst should be generated “in situ” from inexpensive ruthenium compounds and the olefin but the above-described activating agents are dispensed with.

The object is achieved by a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out.

For the purposes of the present invention, the term olefin relates to all types of olefins regardless of whether they are monoolefins or diolefins, cyclic olefins or acyclic internal olefins or acyclic terminal olefins or else mixtures of olefins.

An acyclic internal olefin is an olefin whose C—C double bond is not located on the alpha-carbon.

A terminal olefin is an olefin whose C—C double bond is located on the alpha-carbon.

A diolefin is an olefin having two C—C double bonds in a molecule, with a terminal diolefin having the C—C double bonds on the alpha- and omega-carbon atoms and an internal diolefin having the C—C double bonds neither on the alpha-carbon atom nor on the omega-carbon atom. The olefins can also be substituted. Examples of olefins are the monoolefins and diolefins of the general formulae (a)-(g), where n can independently be 1, 2, 3, 4, 5, 6, 7, 8, 9, 10, 11 or 12 and from 1 to 4 carbon atoms may each be replaced by a heteroatom selected from the group consisting of N, O, 5, P and Si and the hydrogen atoms of the radicals (CₙH₂ₙ)/(CₙH₂ₙ₊₁), NH, PH, POH and SiH₂ may be substituted.

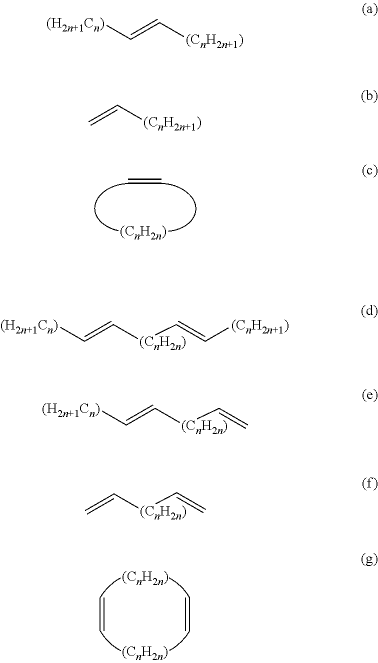

The substituents of the olefins are selected from the group consisting of

—{C₁-C₂₀}-alkyl,
—{C₃-C₈}-cycloalkyl,
—{C₃-C₇}-heterocycloalkyl,
—{C₆-C₁₄}-aryl,
—{C₃-C₁₄}-heteroaryl,
—{C₆-C₁₄}-aralkyl,
—{C₁-C₂₀}-alkyloxy,
—{C₆-C₁₄}-aryloxy,
—{C₆-C₁₄}-aralkyloxy,
—{C₁-C₂₀}-alkylthio,
—{C₆-C₁₄}-arylthio,
—{C₆-C₁₄}-aralkylthio,
—{C₁-C₂₀}-acyl,
—{C₁-C₈}-acyloxy,

### —OH,

### —NH₂,

—NH({C₁-C₂₀}-alkyl),
—NH({C₆-C₁₄}-aryl),
—NH({C₆-C₁₄}-aralkyl),
—NH({C₁-C₈}-acyl),
—NH({C₁-C₈}-acyloxy)
—N((C₁-C₂₀)-alkyl)₂,
—N({C₆-C₁₄}-aryl)₂,
—N({C₆-C₁₄}-aralkyl)₂,
—N({C₁-C₂₀}-alkyl)({C₆-C₁₄}-aryl),
—N({C₁-C₈}-acyl)₂,

### —NH₃⁺,

—NH({C₁-C₂₀}-alkyl)₂⁺,
—NH({C₆-C₁₄}-aryl)₂⁺,
—NH({C₆-C₁₄}-aralkyl)₂⁺,
—NH({C₁-C₂₀}-alkyl)({C₆-C₁₄}-aryl)⁺,
—N({C₆-C₁₄}-aryl)ₓ({C₁-C₂₀}-alkyl)₃₋ₓ⁺,

### —NO₂,

—O—C(O)—O—{C₁-C₂₀}-alkyl,
—O—C(═O)—O—{C₆-C₁₄}-aryl,
—O—C(═O)—O—{C₆-C₁₄}-aralkyl,
—NH—C(═O)—O—{C₁-C₂₀}-alkyl,
—NH—C(═O)—O—{C₆-C₁₄}-aryl,
—NH—C(═O)—O—{C₆-C₁₄}-aralkyl,
—O—C(═O)—NH—{C₁-C₂₀}-alkyl,
—O—C(═O)—NH—{C₆-C₁₄}-aryl,
—O—C(═O)—NH—{C₆-C₁₄}-aralkyl,

### —NH—C(═O)—NH₂,

—NH—C(═O)—NH—{C₁-C₂₀}-alkyl,
—NH—C(═O)—NH—{C₆-C₁₄}-aryl,
—NH—C(═O)—NH—{C₆-C₁₄}-aralkyl,

### —CN,

-halogen,
—C(═N—{C₁-C₂₀}-alkyl)-{C₁-C₂₀}-alkyl,
—C(═N—{C₆-C₁₄}-aryl)-{C₁-C₂₀}-alkyl,
—C(═N—{C₁-C₂₀}-alkyl)-{C₆-C₁₄}-aryl,
—C(═N—{C₆-C₁₄}-aryl)-{C₆-C₁₄}-aryl,
—SO₂—O—{C₁-C₂₀}-alkyl,
—SO₂—O—{C₆-C₁₄}-aryl,
—SO₂—O—{C₆-C₁₄}-aralkyl,
—SO₂—{C₁-C₂₀}-alkyl,
—SO₂—{C₆-C₁₄}-aryl,
—SO₂—{C₆-C₁₄}-aralkyl,
—SO—{C₁-C₂₀}-alkyl,
—SO—{C₆-C₁₄}-aryl,
—SO—{C₆-C₁₄}-aralkyl,
—Si({C₁-C₂₀}-alkyl)₃,
—Si({C₆-C₁₄}-aryl)₃,
—Si({C₆-C₁₄}-aryl)ₓ ({C₁-C₂₀}-alkyl)₃₋ₓ,
{C₁-C₂₀}-perfluoroalkyl,
—PO(O—{C₁-C₂₀}-alkyl)₂,
—PO(O—{C₆-C₁₄}-aryl)₂,
—PO(O—{C₁-C₂₀}-alkyl)(O—{C₆-C₁₄}-aryl),
—PO({C₁-C₂₀}-alkyl)₂,
—PO({C₆-C₁₄}-aryl)₂,
—PO({C₁-C₂₀}-alkyl)({C₆-C₁₄}-aryl).

Examples of monoolefins and diolefins are the following compounds

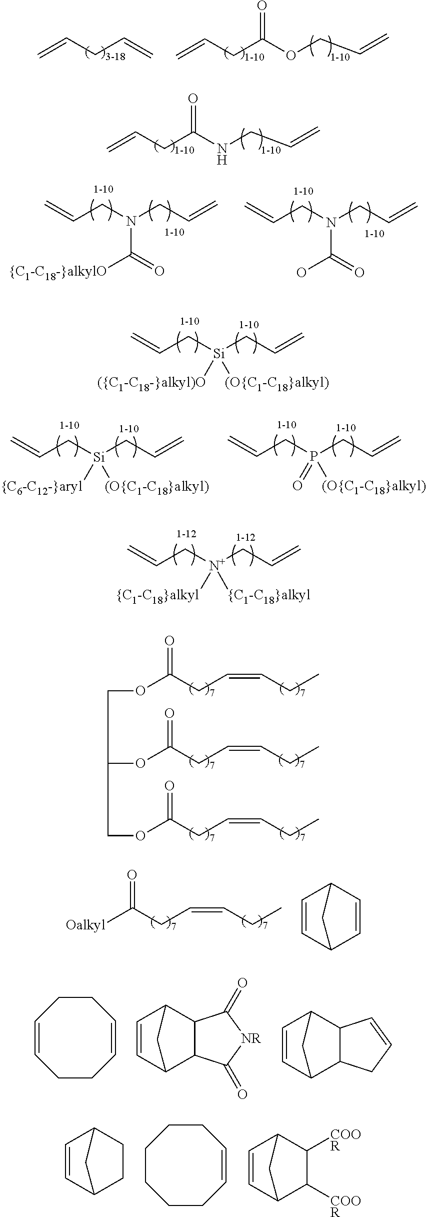

where R is (C₁-C₁₈)-alkyl or (C₆-C₁₂)-aryl and the olefins may be unsubstituted or substituted as described above.

Preference is given to using terminal monoolefins or terminal diolefins which may be unsubstituted or substituted as described above.

The olefins are particularly preferably selected from the group consisting of the following compounds, which may be unsubstituted or substituted as described above.

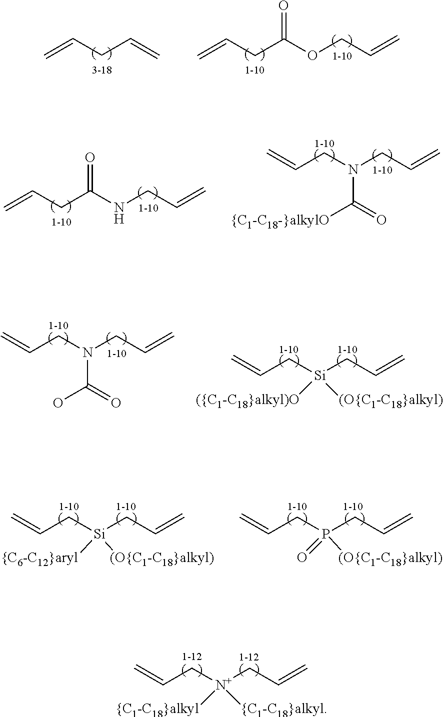

For the purposes of the present invention, anionic ligands are singularly or multiply negatively charged ligands which are selected independently from the group consisting of halide, pseudohalide, tetraphenylborate, hexahalophosphate, methanesulphonate, trihalomethanesulphonate, arylsulphonate, alkoxide, aryloxide, carboxylate, sulphate and phosphate. Pseudohalides are ligands which behave chemically similarly to the halides; among the pseudohalides, preference is given to cyanide (CN⁻), cyanate (OCN⁻), thiocyanate or (SCN⁻). Preference is given to anionic ligands selected independently from the group consisting of the halides fluoride, chloride, bromide and iodide, with chloride being particularly preferred.

For the purposes of the present invention, uncharged i-bonding ligands are monocyclic and polycyclic arenes which may also have substituents which may be identical or nonidentical, where the substituents are selected from the group consisting of (C₁-C₂₀)-alkyl, (C₆-C₁₄)-aryl, (C₁-C₂₀)-alkyloxy, (C₆-C₁₄)-aryloxy, (C₁-C₂₀)-perfluoroalkyl, (C₁-C₂₀)-alkylthio, (C₂-C₁₀)-alkenylthio, (C₂-C₁₀)-alkenyl, (C₂-C₁₀)-alkynyl, (C₂-C₁₀)-alkenyloxy, (C₂-C₁₀)-alkynyloxy, and halogen. The substituents can in turn likewise be substituted, where these substituents are selected from the group consisting of halogen, (C₁-C₈)-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₈)-alkyl, —SO—(C₆-C₁₄)-aryl, —PO(O—{C₁-C₂₀}-alkyl)₂, —PO(O—{C₆-C₁₄}-aryl)₂, —PO(P—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl). Examples are benzene, toluene, xylene, cymene, trimethylbenzene, tetramethylbenzene, hexamethylbenzene, tetrahydronaphthalene, and naphthalene. The uncharged i-bonding ligand is particularly preferably selected from the group consisting of benzene, cymene and hexamethylbenzene.

For the purposes of the present invention, an uncharged electron donor ligand is a ligand which does not have a net charge and makes available free electron pairs or electron-filled orbitals for a coordinate bond to an acceptor. An acceptor is an atom which can take up free electrons or electrons from a filled orbital from the donor. Donors are typically main group elements of groups 13-17 of the Periodic Table of the Elements, e.g. C, N, P. Even carbon can occur as uncharged electron donor. Carbon most frequently occurs as uncharged electron donor in carbenes, where the carbon atom bears an electron pair in an orbital. These electrons are available for a sigma-bond to an acceptor atom. Acceptors are typically metal atoms such as Pd(0), Pd(II), Ru (I) or Ru(II). Typical examples of uncharged electron donor ligands are heterocyclic carbenes, phosphanes, phosphinites, phosphonites, phosphites, arsanes and nitrogen bases.

The uncharged electron donor ligand is particularly preferably selected from the group consisting of phosphanes and N-heterocyclic carbenes. Preference is given to N-heterocyclic carbenes selected from the group consisting of compounds of the formulae VI-XI and phosphanes selected from the group consisting of P(phenyl)₃ and P(cyclohexyl)₃.

For the purposes of the present invention, a heterocyclic carbene is a carbene of the general formula (V)

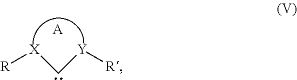

where
R and R′ are identical or different and are selected from the group consisting of
hydrogen,
(C₁-C₁₈)-alkyl,
(C₃-C₈)-cycloalkyl,
(C₃-C₇)-heterocycloalkyl,
(C₆-C₁₄)-aryl,
and (C₃-C₁₄)-heteroaryl;
X and Y are selected independently from the group consisting of carbon, nitrogen and phosphorus atoms and
A is a (C₂-C₄)-alkylene bridge or a (C₂-C₄)-heteroalkylene bridge.

Examples of heterocyclic carbenes are the N-heterocyclic carbenes of the formulae VI to XI:

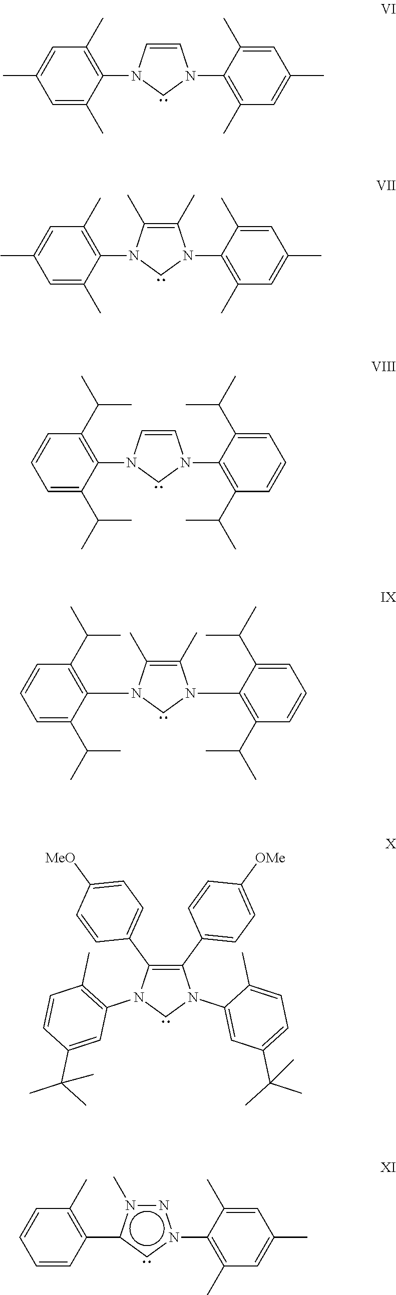

N-heterocyclic carbenes can be obtained in situ by thermal activation of carbene adducts or the combination of an N-heterocyclic carbene precursor and a base. As base, it is possible to use any inorganic or organic base, with preference being given to using nitrogen bases or an alkyloxy base. Particular preference is given to alkyloxy bases which are selected from the group consisting of sodium and potassium salts of methoxide, ethoxide, propoxide and butoxide and isomers thereof. Typical examples of carbene adducts are adducts of N-heterocyclic carbenes with alcohols, chloroform, pentafluorophenol, CO₂ and borane.

For the purposes of the present invention, phosphanes are compounds of the general formula PR¹R²R³,

where R¹, R² and R³ can be identical or different and can be selected from the group consisting of H, (C₁-C₁₈)-alkyl, (C₃-C₈)-cycloalkyl, (C₂-C₇)-heterocycloalkyl, (C₆-C₁₄)-aryl and (C₃-C₁₄)-heteroaryl, where the radicals R¹, R² and R³ can optionally have one or more cyclic structures. Typical examples are P(phenyl)₂, P(cyclohexyl)₂, P(isopropyl)₂, P(cyclopentyl)₂, P(tert-butyl)₂, P(neopentyl)₂, with particular preference being given to P(phenyl)₃, P(cyclohexyl)₃ and P(isopropyl)₃.

For the purposes of the present invention, phosphinites are compounds of the general formula PR^{1′}R^{2′}(OR^{3′}), where R^{1′} and R^{2′} can be identical or different and can be selected from the group consisting of (C₁-C₁₈)-alkyl, (C₃-C₈)-cycloalkyl, (C₂-C₇)-heterocycloalkyl, (C₆-C₁₄)-aryl and (C₃-C₁₄)-heteroaryl and R^{3′} can be selected from the group consisting of H, (C₁-C₈-alkyl, (C₆-C₁₄)-aryl, where the radicals R¹, R² and R^{3′} can optionally have one or more cyclic structures. Typical examples are (tert-butyl)₂P(Obutyl), (1-adamantyl)₂P(Obutyl), Ph₂P(OEt), Ph₂P(OPh), P(OPh)₂, Ph₂P(O-(2,4-di-tert-butyl)phenyl).

For the purposes of the present invention, phosphonites are compounds of the general formula P(OR^{1″})(OR^{2″})R^{3″}, where R^{1″} and R^{2″} can be identical or different and can be selected from the group consisting of (C₁-C₈)-alkyl, (C₆-C₁₄)-aryl, and R^{3″} can be selected from the group consisting of H, (C₁-C₁₈)-alkyl, (C₃-C₈)-cycloalkyl, (C₂-C₇)-heterocycloalkyl, (C₆-C₁₄)-aryl and (C₃-C₁₄)-heteroaryl, where the radicals R^{1″}, R^{2″} and R^{3″} can optionally have one or more cyclic structures. Typical examples are MeP(OMe)₂, EtP(OEt)₂, PhP(OEt)₂, PhP(OPh)₂, PhCH₂P(OPh)₃ and PhP(O-(2,4-di-tert-butyl)phenyl)₂.

For the purposes of the present invention, phosphites are compounds of the general formula P(OR^{1′″})(OR^{2′″})(OR^{3′″}), where R^{1′″}, R^{2′″} and R^{3′″} can be identical or different and can be selected from the group consisting of H, (C₁-C₈)-alkyl, (C₆-C₁₄)-aryl, where the radicals R^{1′″}, R^{2′″} and R^{3′″} can optionally have one or more cyclic structures. Typical examples are P(Omethyl)₃, P(Oethyl)₃, P(Ophenyl)₃, P(O-(2,4-di-tert-butyl)phenyl)₃ and

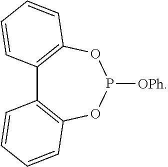 [SMILES: c1ccc(Op2oc3ccccc3c3ccccc3o2)cc1]

For the purposes of the present invention, arsanes are compounds of the general formula AsR^{1″″}R^{2″″}R^{3″″}, where R^{1″″}, R^{2″″} and R^{3″″} can be identical or different and can be selected from the group consisting of H, (C₁-C₁₈)-alkyl, (C₃-C₈)-cycloalkyl, (C₂-C₇)-heterocycloalkyl, (C₆-C₁₄)-aryl and (C₃-C₁₄)-heteroaryl, where the radicals R¹, R² and R³ can optionally have one or more cyclic structures. Typical examples are As(phenyl)₃, As(cyclohexyl)₃, As(isopropyl)₃, As(cyclopentyl)₃, As(tert-butyl)₃, As(neopentyl)₃, with particular preference being given to As(phenyl)₃ᵣ As(cyclohexyl)₃ and As(isopropyl)₃.

For the purposes of the present invention, nitrogen bases are amines and nitrogen aromatics. Examples of amines are ammonia, triethylamine, N,N-dimethylaniline, piperidine, N-methyl-pyrrolidine, 1,4-diazabicyclo[2.2.2]octane or 1,8-diazobicyclo[5.4.0]undec-7-ene, with particular preference being given to triethylamine. Examples of nitrogen aromatics are pyridine, pyrimidine, pyrazine, pyrrole, indole, carbazole, imidazole, pyrazole, benzimidazole, oxazole, triazole, isoxazole, isothiazole, triazole, quinoline, isoquinoline, acridine, phenazine, phenoxazine, phenothiazine or triazine, with particular preference being given to pyridine.

For the purposes of the present invention (C₁-Cₙ)-alkyl is defined as a linear or branched C₁-Cₙ-alkyl group having from 1 to n carbon atoms. Typical examples of C₁-Cₙ-alkyl groups are methyl, ethyl, n-propyl, isopropyl, n-butyl, isobutyl, sec-butyl, tert-butyl, pentyl, hexyl, heptyl, octyl, nonanyl, decanyl, dodecanyl, or octadecanyl including all their structural isomers. A (C₁-Cₙ)-alkyl group can also be substituted by at least one substituent, with the substituents preferably being selected independently from the group consisting of halogen, (C₁-C₈)-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₈)-alkyl, —SO—(C₆-C₁₄)-aryl, —PO(O—{C₁-C₂₀}-alkyl)₂, —PO(O—{C₆-C₁₄}-aryl)₂, —PO(O—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl).

For the purposes of the present invention, (C₃-Cₙ)-cycloalkyl is a cyclic alkyl group having from 3 to n carbon atoms, with monocyclic, bicyclic and tricyclic alkyl groups being encompassed. Typical examples are cyclopropyl, cyclobutyl, cyclopentyl, cyclohexyl and cycloheptyl. A (C₁-Cₙ)-cycloalkyl group can also be substituted by at least one substituent, with the substituents being selected independently from the group consisting of halogen, (C₁-C₈)-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₈)-alkyl, —SO—(C₆-C₁₄)-aryl, —PO(O—{C₁-C₂₀}-alkyl)₂, —PO(O—{C₆-C₁₄}-aryl)₂, —PO(O—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl).

For the purposes of the present invention, (C₂-Cₙ)-heterocycloalkyl is a cyclic alkyl group having from 2 to n carbon atoms, with monocyclic, bicyclic and tricyclic alkyl groups being encompassed, in which 1 or 2 carbon atoms of the rings have each been replaced by a heteroatom selected from the group consisting of N, O and S. (C₂-Cₙ)-heterocycloalkyl group can also be substituted by at least one substituent, with the substituent being selected independently from the group consisting of halogen, (C₁-C₈)-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₈)-alkyl, —SO—(C₆-C₁₄)-aryl, —PO(O—{C₁-C₂₀}-alkyl)₂, —PO(O—{C₆-C₁₄}-aryl)₂, —PO(O—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl). Typical examples are 2- or 3-tetrahydrofuryl, 1-, 2- or 3-pyrrolidinyl, 1-, 2-, 3- or 4-piperidinyl, 1-, 2-, or 3-morpholinyl, 1-, or 2-piperazinyl, 1-caprolactyl.

For the purposes of the present invention, (C₆-Cₙ)-aryl is a cyclic aromatic group having from 6 to n carbon atoms. In particular, this includes compounds such as phenyl, naphthyl, anthryl, phenanthryl, biphenyl radicals or systems of the above-described type fused onto the molecule concerned, e.g. indenyl systems. A (C₆-Cₙ)-aryl group can also be substituted by at least one substituent, with the substituents being selected independently from the group consisting of halogen, (C₁-C₈)-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₃)-alkyl, —SO—(C₆-C₁₄)-aryl, —PO(O—{C₁-C₂₀}-alkyl)₂, —PO(O—{C₆-C₁₄}-aryl)₂, —PO(O—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl).

For the purposes of the present invention, (C₃-Cₙ)-heteroaryl is a five-, six- or seven-membered aromatic ring system having from 3 to n carbon atoms, where from 1 to 3 carbon atoms of the ring system have each been replaced by a heteroatom selected from the group consisting of N, O and S. Such heteroaryl groups are, in particular, groups such as 2-, 3-furyl, 2-, 3-pyrrolyl, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl. A (C₃-Cₙ)-heteroaryl group can also be substituted by at least one substituent, with the substituents being selected independently from the group consisting of halogen, (C₁-C₈-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₈-alkyl, —SO—(C₆-C₁₄)-aryl, —PO(O—{C₁-C₂₀}-alkyl)₂, —PO(O—{C₆-C₁₄}-aryl)₂, —PO(O—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl).

For the purposes of the present invention, (C₆-Cₙ)-aralkyl is a group which contains both an alkyl group and an aryl group and has a total of from 6 to n carbon atoms. The aralkyl group can be bound via any of its carbon atoms to the molecule bearing this group. A (C₆-Cₙ)-aralkyl group can also be substituted by at least one substituent, with the substituents being selected independently from the group consisting of halogen, (C₁-C₈)-alkyl, (C₁-C₈)-alkyloxy, —NH₂, —NO, —NO₂, NH(C₁-C₈)-alkyl, —N((C₁-C₈)-alkyl)₂, —OH, —CF₃, —CₙF₂ₙ₊₁ (where n is an integer from 2 to 5), NH(C₁-C₈)-acyl, —N((C₁-C₈)-acyl)₂, (C₁-C₈)-acyl, (C₁-C₈)-acyloxy, —SO₂—(C₁-C₈)-alkyl, —SO₂—(C₆-C₁₄)-aryl, —SO—(C₁-C₈)-alkyl, —SO—(C₆-C₁₄)-aryl, —PO(O—{C₁-C₂₀}-alkyl)₂, —PO(O—{C₆-C₁₄}-aryl)₂, —PO(O—{C₁-C₂₀}-alkyl)(O—{C₆-C₁₄}-aryl), —PO({C₁-C₂₀}-alkyl)₂, —PO({C₆-C₁₄}-aryl)₂, —PO({C₁-C₂₀}-alkyl)({C₆-C₁₄}-aryl).

For the purposes of the present invention, (C₂-Cₙ)-alkylene is a divalent linear (C₂-Cₙ)-alkyl group having from 2 to n carbon atoms. Typical examples are ethylene, n-propylene, isopropylene, n-butylene, isobutylene, tert-butylene, n-pentylene, n-hexylene, n-heptylene, n-octylene, n-nonylene, n-decylene. The (C₂-Cₙ)-alkylene group can also have substituents selected from the group consisting of (C₁-C₂₀)-alkyl, (C₆-C₁₄)-aryl, (C₂-C₁₀)-alkenyl. In addition, the (C₂-Cₙ)-alkylene group can be unsaturated, in which case the unsaturated section can be part of an aromatic or heteroaromatic system.

For the purposes of the present invention, (C₂-Cₙ)-heteroalkylene is a divalent linear (C₂-Cₙ)-alkyl group having from 2 to n carbon atoms, where 1 or 2 carbon atoms have been replaced by heteroatoms such as N, O, S. The (C₂-Cₙ)-heteroalkylene group can also have substituents selected from the group consisting of (C₁-C₂₀)-alkyl, (C₆-C₁₄)-aryl, (C₂-C₁₀)-alkenyl. In addition, the (C₂-Cₙ)-heteroalkylene group can be unsaturated, in which case the unsaturated section can be part of an aromatic or heteroaromatic system.

For the purposes of the present invention, (C₂-Cₙ)-alkenyl is defined as a linear or branched (C₂-Cₙ)-alkyl group having from 2 to n carbon atoms, with the proviso that it has a C—C double bond.

For the purposes of the present invention, (C₂-Cₙ)-alkynyl is defined as a linear or branched (C₂-Cₙ)-alkyl group having from 2 to n carbon atoms, with the proviso that it has a C—C triple bond.

For the purposes of the present invention, (C₁-Cₙ)-alkyloxy is defined as a linear or branched C₁-Cₙ-alkyl group having from 1 to n carbon atoms, with the proviso that it is bound via an oxygen atom to the molecule bearing this group.

For the purposes of the present invention, (C₂-Cₙ)-alkenyloxy is defined as a linear or branched C₂-Cₙ-alkenyl group having from 2 to n carbon atoms, with the proviso that it is bound via an oxygen atom to the molecule bearing this group.

For the purposes of the present invention, (C₂-Cₙ)-alkynyloxy is defined as a linear or branched C₂-Cₙ-alkynyl group having from 2 to n carbon atoms, with the proviso that it is bound via an oxygen atom to the molecule bearing this group.

For the purposes of the present invention, (C₁-Cₙ)-acyl is a group having the general structure R—(C═O)— and a total of from 1 to n carbon atoms, where R is selected from the group consisting of H, (C₁-Cₙ₋₁)-alkyl, (C₁-Cₙ₋₁)-alkenyl, (C₆-Cₙ₋₁)-aryl, (C₆-Cₙ₋₁)-heteroaryl and (C₂-Cₙ₋₁)-alkynyl.

For the purposes of the present invention, (C₁-Cₙ)-acyloxy is a group having the general structure R—(C═O)O— and a total of from 1 to n carbon atoms, where R is selected from the group consisting of H, (C₁-Cₙ₋₁)-alkyl, (C₁-Cₙ₋₁)-alkenyl, (C₆-Cₙ₋₁)-aryl, (C₆-Cₙ₋₁)-heteroaryl and (C₂-Cₙ₋₁)-alkynyl.

For the purposes of the present invention, a noncoordinating salt is an inorganic salt selected from the group consisting of a sodium, potassium, caesium, barium, calcium or magnesium salt of PF₆⁻, BF₄⁻, BH₄⁻, F₃CSO₃⁻, H₃CSO₃⁻, ClO₄⁻, SO₄²⁻, HSO₄⁻, NO₃⁻, PO₄³⁻, HPO₄²⁻, H₂PO₄⁻, CF₃COO⁻, B(C₆F₅)₄⁻, B[3,5-(CF₃)₂C₆H₃]₄⁻, RSO₃⁻ and R′COO⁻, where R, R′ are selected independently from the group consisting of (C₁-C₂₀)-alkyl and (C₆-C₁₄)-aryl. R is preferably selected from the group consisting of methyl, phenyl, p-tolyl and p-nitrophenyl, and R′ is preferably selected from the group consisting of H, methyl, phenyl, p-tolyl and p-nitrophenyl.

The noncoordinating salt is preferably selected from the group consisting of a sodium, potassium, caesium, barium, calcium or magnesium salt of PF₆⁻, BF₄⁻, F₃CSO₃⁻, CF₃COO⁻, B(C₆F₅)₄⁻; the noncoordinating salt is particularly preferably NaPF₆ or KPF₆.

For the purposes of the present invention, a Lewis acid is a compound selected from the group consisting of aluminium, boron, chromium, cobalt, iron, copper, magnesium, lanthanum, magnesium, nickel, palladium and zinc salts of Cl—, Br—, I—, PF₆—, BF₄—, CF₃COO—, B(C₆F₅)₄—, B[3,5-(CF₃)₂C₆H₃]₄—, R^{x}COO—, (R^{y}COCHCOR^{z})—, where R^{x}, R^{y}, R^{z} are selected independently from the group consisting of (C₁-C₂₀)-alkyl and (C₆-C₁₄)-aryl. R^{x}, R^{y}, R^{z} are preferably selected independently from the group consisting of methyl and phenyl. The Lewis acid is particularly preferably selected from the group consisting of aluminium and iron salts of Cl—, MeCOO— or (MeCOCHCOMe)-, with particular preference being given to aluminium(III) chloride, iron(II) acetate and iron(III) acetylacetonate.

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and a halogen compound is additionally added in step c).

For the purposes of the present invention, a halogen compound is a compound selected from the group consisting of salt-like halides and organic halogen compounds. Salt-like halides are selected from the group consisting of lithium, sodium, potassium, caesium, magnesium, calcium and ammonium halides, where ammonium halides are compounds of the general formula [NHₓ{(C₁-C₂₀)-alkyl)₄₋ₓ]⁺[halide]⁻ and the halides are selected from the group consisting of chloride, bromide and iodide. An organic halogen compound is a monohalogenated or dihalogenated (C₁-C₂₀)-alkyl, (C₃-C₁₀)-cycloalkyl, (C₆-C₁₄)-aryl or (C₁-C₂₀)-aralkyl compound, where the halogens are selected independently from the group consisting of chlorine, bromine and iodine. The organic halogen compound is preferably selected from the group consisting of monobromo and dibromo compounds, aromatic 1,2-organodihalides and allylic monobromides. The salt-like halide is preferably selected from the group consisting of sodium bromide, sodium iodide, potassium chloride, potassium bromide, potassium iodide and tetraalkylammonium bromides. The halogen compound is preferably selected from the group consisting of potassium iodide, potassium bromide, tetrabutylammonium bromide, 1,2-dibromocyclohexane, 1,2-bromocyclohexane, 1,2-dibromoethane, 1,2-dibromo-4,5-dimethylbenzene, 1,2-diiodobenzene, 1-bromo-2-iodobenzene, 2-bromostyrene, (2-bromoethyl)benzene and (3-bromopropyl)benzene.

The olefin metathesis reactions are usually carried out by bringing an olefin or an olefin mixture into contact with a ruthenium compound of the general formula (I), adding a Lewis acid or adding a Lewis acid and an anionic, noncoordinating salt and optionally adding a halogen compound and subsequently heating the reaction mixture until the reaction is complete, so that the catalytically active ruthenium compound is formed “in situ”. The reaction temperature is in the range from 30° C. to 140° C., preferably in the range from 40° C. to 140° C., more preferably in the range from 60° C. to 100° C. and particularly preferably in the range from 70° C. to 100° C.

The reaction time is not critical and is in the range from a few minutes to some hours, preferably in the range from 30 minutes to 3 hours.

The reactions are generally carried out under a protective gas atmosphere, preferably under nitrogen or argon, although the presence of oxygen can be tolerated under particular circumstances. The reactions can, under particular circumstances, be carried out in the presence of water. The metathesis reactions can be carried out in all solvents or solvent mixtures which do not deactivate the catalyst. Preference is given to selecting aprotic solvents having little tendency to coordinate. If the olefin is liquid, the reaction can be carried out without a solvent. Solvents are preferably selected from the group consisting of dichloromethane, 1,2-dichlorethane, benzene, toluene, xylene, halobenzene and hexane.

In metathesis reactions the ratio of ruthenium compound to olefin is not critical and is in the range from 1:10 to 1:1 000 000. In the ring-closing metathesis reaction, the ratio of ruthenium compound to olefin is preferably in the range from 1:100 to 1:10 000. In the cross-metathesis reaction, the ratio of ruthenium compound to olefin is preferably in the range from 1:10 to 1:100 and in the ring-opening polymerization metathesis reaction the ratio of ruthenium compound to olefin is preferably in the range from 1:1000 to 1:1 000 000.

The ratio of ruthenium compound to anionic, noncoordinating salt is in the range from 1:1 to 1:10; the ratio is preferably in the range from 1:1 to 1:5 and particular preference is given to a ratio of 1:5.

The ratio of ruthenium compound to Lewis acid is in the range from 1:1 to 1:10; the ratio is preferably in the range from 1:1 to 1:5 and particular preference is given to a ratio of 1:5.

The ratio of ruthenium compound to halogen compound is in the range from 1:1 to 1:333; the ratio is preferably in the range from 1:1 to 1:33.

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is selected from among compounds belonging to the group consisting of the following elements (a) compounds derived from formula (I) in which x=1, y=1, z=1 (formula II), (b) compounds derived from formula (I) in which x=0, y=2, z=1 (formula III) and (c) compounds derived from formula (I) in which x=0, y=1, z=2 (formula IV)

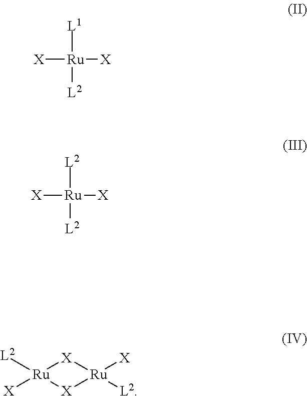

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula RuX₂L¹L² (II), where the ligands X are identical and are each chlorine and L² is selected from the group consisting of N-heterocyclic carbenes and phosphanes.

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula RuX₂L¹L² (II) where the ligands X are identical and are each chlorine, L¹ is selected from the group consisting of benzene, toluene, xylene, cymene, trimethylbenzene, tetramethylbenzene, hexamethylbenzene, tetrahydronaphthalene and naphthalene and L² is selected from the group consisting of P(cyclohexyl)₃ and the N-heterocyclic carbenes of the formulae VI, VII, VIII, IX, X and XI.

Preferred ruthenium compounds of the general formula (II) are shown in Table 1.

TABLE 1
Preferred ruthenium compounds of the general formula (II)

| Ru | | | |
|---|---|---|---|
| compound | X | L¹ | L² |
| 1 | Cl | Benzene | P(phenyl)₃ |
| 2 | Cl | Benzene | P(cyclohexyl)₃ |
| 3 | Cl | Benzene | VI |
| 4 | Cl | Benzene | VII |
| 5 | Cl | Benzene | VIII |
| 6 | Cl | Benzene | IX |
| 7 | Cl | Benzene | X |
| 8 | Cl | Benzene | XI |
| 9 | Cl | Cymene | P(phenyl)₃ |
| 10 | Cl | Cymene | P(cyclohexyl)₃ |
| 11 | Cl | Cymene | VI |
| 12 | Cl | Cymene | VII |
| 13 | Cl | Cymene | VIII |
| 14 | Cl | Cymene | IX |
| 15 | Cl | Cymene | X |
| 16 | Cl | Cymene | XI |
| 17 | Cl | Hexamethylbenzene | P(phenyl)₃ |
| 18 | Cl | Hexamethylbenzene | P(cyclohexyl)₃ |
| 19 | Cl | Hexamethylbenzene | VI |
| 20 | Cl | Hexamethylbenzene | VII |
| 21 | Cl | Hexamethylbenzene | VIII |
| 22 | Cl | Hexamethylbenzene | IX |
| 23 | Cl | Hexamethylbenzene | X |
| 24 | Cl | Hexamethylbenzene | XI |

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula RuX₂L¹L² (II) and is selected from the group consisting of the compounds of the formulae A, B and C

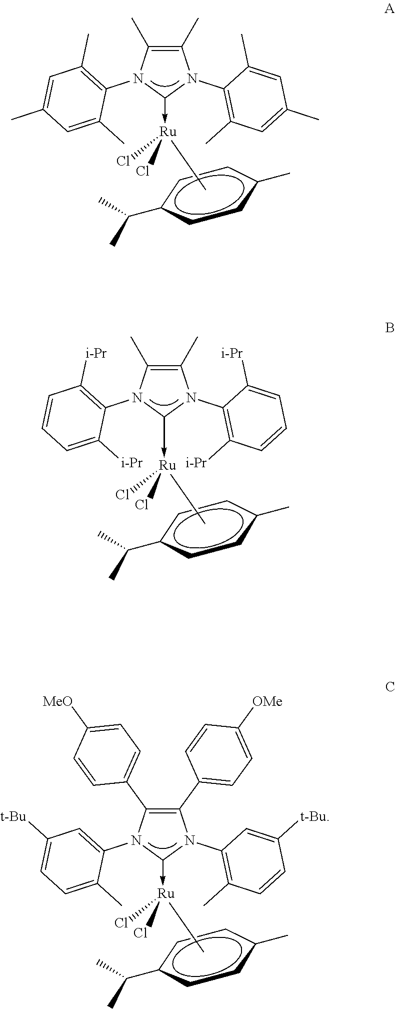

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula RuX₂L²₂ (III), where the ligands X are identical and are each chlorine and the ligands L² are selected independently from the group consisting of N-heterocyclic carbenes and phosphanes.

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula RuX₂L²₂ (III), where the ligands X are identical and are each chlorine and the ligands L² are selected independently from the group consisting of P(cyclohexyl)₃, P(phenyl)₃ and the N-heterocyclic carbenes of the formulae VI, VII, VIII, IX, X and XI, with the proviso that the ligands L² are not both P(phenyl)₃.

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula RuX₂L²₂ (III), where the ligands X are identical and are each chlorine and one L² is selected from the group consisting of P(cyclohexyl)₃ and P(phenyl)₃ and the other L² is selected from the group consisting of the N-heterocyclic carbenes of the formulae VI, VII, VIII, IX, X and XI.

Preferred ruthenium compounds of the general formula (III) are shown in Table 2.

TABLE 2
Preferred ruthenium compounds of the general formula (III)

| Ru | | | |
|---|---|---|---|
| compound | X | L² | L² |
| 25 | Cl | P(phenyl)₃ | P(cyclohexyl)₃ |
| 26 | Cl | P(phenyl)₃ | VI |
| 27 | Cl | P(phenyl)₃ | VII |
| 28 | Cl | P(phenyl)₃ | VIII |
| 29 | Cl | P(phenyl)₃ | IX |
| 30 | Cl | P(phenyl)₃ | X |
| 31 | Cl | P(phenyl)₃ | XI |
| 32 | Cl | P(cyclohexyl)₃ | P(cyclohexyl)₃ |
| 33 | Cl | P(cyclohexyl)₃ | VI |
| 34 | Cl | P(cyclohexyl)₃ | VII |
| 35 | Cl | P(cyclohexyl)₃ | VIII |
| 36 | Cl | P(cyclohexyl)₃ | IX |
| 37 | Cl | P(cyclohexyl)₃ | X |
| 38 | Cl | P(cyclohexyl)₃ | XI |
| 39 | Cl | VI | VI |
| 40 | Cl | VI | VII |
| 41 | Cl | VI | VIII |
| 42 | Cl | VI | IX |
| 43 | Cl | VI | X |
| 44 | Cl | VI | XI |
| 45 | Cl | VII | VII |
| 46 | Cl | VII | VIII |
| 47 | Cl | VII | IX |
| 48 | Cl | VII | X |
| 49 | Cl | VII | XI |
| 50 | Cl | VIII | VIII |
| 51 | Cl | VIII | IX |
| 52 | Cl | VIII | X |
| 53 | Cl | VIII | XI |
| 54 | Cl | IX | IX |
| 55 | Cl | IX | X |
| 56 | Cl | IX | XI |
| 57 | Cl | X | X |
| 58 | Cl | X | XI |
| 59 | Cl | XI | XI |

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula [RuX₂L²]₂ (IV), where the ligands X are identical and are each chlorine and the ligands L² are selected independently from the group consisting of N-heterocyclic carbenes and phosphanes.

The invention further provides a process for preparing olefins by means of metathesis, which comprises the following steps
a) provision of an olefin reaction mixture containing at least one olefin,
b) addition of a ruthenium compound of the general formula [RuX₂L¹ₓL²_{y}]_{z} (I), where X=anionic ligand; L¹=uncharged π-bonding ligand; L²=uncharged electron donor ligand; x=0, 1; y=1, 2, 3; z=1, 2,
c) addition of a Lewis acid or addition of a Lewis acid and an anionic, noncoordinating salt,
d) reaction at temperatures in the range from 30° C. to 140° C.,
   where no addition of an alkyne or alkynol is carried out and the ruthenium compound is a compound of the general formula [RuX₂L²]₂ (IV), where the ligands X are identical and are each chlorine and the ligands L² are identical and are selected from the group consisting of P(cyclohexyl)₃ and the N-heterocyclic carbenes of the formulae VI, VII, VIII, IX, X and XI.

Preferred ruthenium compounds of the general formula (IV) are shown in Table 3.

TABLE 3
Preferred ruthenium compounds of the general formula (IV)

| Ru | | | |
|---|---|---|---|
| compound | X | L² | L² |
| 60 | Cl | P(cyclohexyl)₃ | P(cyclohexyl)₃ |
| 61 | Cl | P(cyclohexyl)₃ | VI |
| 62 | Cl | P(cyclohexyl)₃ | VII |
| 63 | Cl | P(cyclohexyl)₃ | VIII |
| 64 | Cl | P(cyclohexyl)₃ | IX |
| 65 | Cl | P(cyclohexyl)₃ | X |
| 66 | Cl | P(cyclohexyl)₃ | XI |
| 67 | Cl | VI | VI |
| 68 | Cl | VI | VII |
| 69 | Cl | VI | VIII |
| 70 | Cl | VI | IX |
| 71 | Cl | VI | X |
| 72 | Cl | VI | XI |
| 73 | Cl | VII | VII |
| 74 | Cl | VII | VIII |
| 75 | Cl | VII | IX |
| 76 | Cl | VII | X |
| 77 | Cl | VII | XI |
| 78 | Cl | VIII | VIII |
| 79 | Cl | VIII | IX |
| 80 | Cl | VIII | X |
| 81 | Cl | VIII | XI |
| 82 | Cl | IX | IX |
| 83 | Cl | IX | X |
| 84 | Cl | IX | XI |
| 85 | Cl | X | X |
| 86 | Cl | X | XI |
| 87 | Cl | XI | XI |

The invention further provides for the use of the process in metathesis reactions selected from the group consisting of cross-metathesis (CM), ring-closing metathesis (RCM), ring-opening metathesis (ROM), ring-opening metathesis polymerization (ROMP) and acyclic diene metathesis (ADMET). Preference is given to ring-closing metathesis (RCM) and cross-metathesis (CM).

In contrast to the ruthenium and osmium complexes described in EP0921129A1 for olefin metathesis, no isolation of the ionic complexes and also no metal-carbene unit of the general formula M=C(═C)ₙ═CR¹R² are necessary in the present inventive process in order to achieve activity of the ruthenium compound. The use of alkynols such as the toxic propargyl alcohol can therefore be dispensed with in the olefin metathesis process of the invention. In addition, no activating agents such as photochemical activation, disubstituted cyclopropenes, diazoalkanes or alkynes are necessary to achieve activity of the ruthenium compound.

Furthermore, it is possible to use the ruthenium compound in an amount of less than 1 mol %, based on the olefin.

### EXPERIMENTAL PART

The following ruthenium compounds were used for the metathesis reactions:

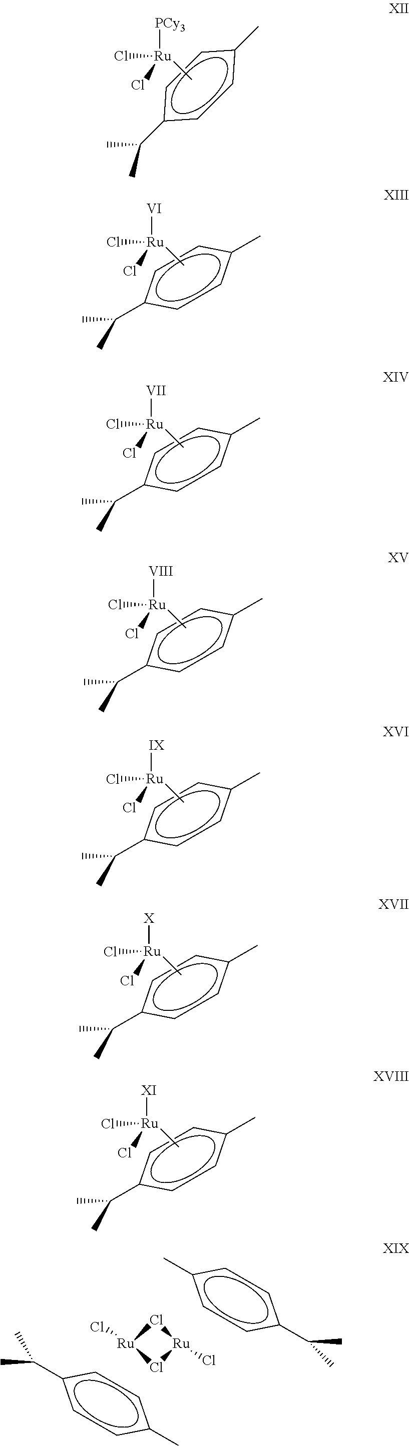

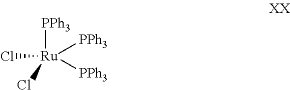

The examples below serve to illustrate the process of the invention without restricting it thereto.

### General

The reactions and the production of stock solutions on the air-sensitive compounds were carried out in an argon-filled glove box or in standard Schlenk flasks and Schlenk apparatuses. All solvents were dried and stored over molecular sieves. The commercially available metal salts and halogen compounds were used without further purification. The noncoordinating salts were dried if necessary and stored and also dispensed, if they are hygroscopic, in a glove box. The olefins and the internal standard were if necessary degassed and dried over molecular sieves. All ring-closing metathesis products and cross-metathesis products are known; they were determined quantitatively relative to hexadecane by means of gas chromatography.

### Examples 1 to 24

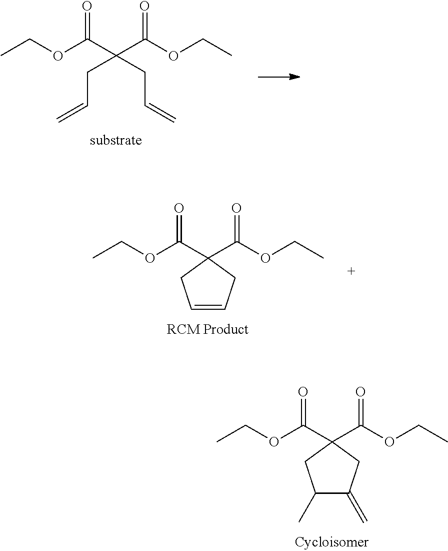 [SMILES: C=CCC(CC=C)(C(=O)OCC)C(=O)OCC ; CCOC(=O)C1(C(=O)OCC)CC=CC1 ; C=C1CC(C(=O)OCC)(C(=O)OCC)CC1C]

Ruthenium compounds of the general formulae XII-XX were examined to determine their activity and selectivity in the ring-closing metathesis reaction of diethyl diallylmalonate to form diethyl cyclopent-3-ene-1,1-dicarboxylate relative to the cycloisomerization reaction. The results are summarized in Table 4.

### Example 1

### Comparative Experiment

A 250 ml three-neck flask was charged with 961.2 mg (4.00 mmol) of diethyl diallylmalonate, 452.6 mg (2.00 mmol) of hexadecane and 100 ml of dry toluene and the reaction mixture was heated to 80° C. in a stream of argon. In a glove box, 25.5 mg (0.04 mmol) of the ruthenium compound XIV were dissolved in 8 ml of dry toluene and introduced into the hot reaction solution in the 250 ml flask. The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). In addition, the solvent was distilled off from the reaction mixture and the residue was fractionated by means of column chromatography to give 271.0 mg of diethyl cyclopent-3-ene-1,1-dicarboxylate (yield isolated: 32.0%).

### Examples 2 to 9, 14 and 19 to 24

A 250 ml three-neck flask was charged with 4.00 mmol of diethyl diallylmalonate, 2.00 mmol of hexadecane, 0 mmol-0.20 mmol of Lewis acid (AlCl₃, PdCl₂(PPh₃)₂, Fe(OAc)₂ or Fe(acac)₃), 0 mmol-0.20 mmol of NaPF₆ and 100 ml of dry solvent (toluene or xylene) and the reaction mixture was heated to 80-140° C. in a stream of argon. In a glove box, 0.04 mmol of the ruthenium compound (XII-XVIII) was dissolved in 8 ml of dry toluene and introduced into the hot reaction solution in the 250 ml flask. The reaction solution was stirred at the desired reaction temperature for 1-3 hours in a stream of argon. After the reaction was complete, 0.1 ml of the reaction solution was dissolved in 1.5 ml of ethyl acetate and analysed by means of an HP 6890 gas chromatograph (GC). The specific reaction conditions and results are shown in Table 4.

### Examples 10 to 13

A 250 ml three-neck flask was charged with 4.00 mmol of diethyl diallylmalonate, 2.00 mmol of hexadecane, 0.20 mmol of Fe(acac)₃, 0.20 mmol of NaPF₆, 0.04 mmol of 1,2-dibromocyclohexane and 100 ml of dry toluene and the reaction mixture was heated to 40-100° C. in a stream of argon. In a glove box, 0.04 mmol of the ruthenium compound XIV was dissolved in 8 ml of dry toluene and introduced into the hot reaction solution in the 250 ml flask. The reaction solution was stirred at the desired reaction temperature for 3 hours in a stream of argon. After the reaction was complete, 0.1 ml of the reaction solution was dissolved in 1.5 ml of ethyl acetate and analysed by means of an HP 6890 gas chromatograph (GC). The specific reaction conditions and results are shown in Table 4.

### Examples 15 to 18

A 250 ml three-neck flask was charged with 4.00 mmol of diethyl diallylmalonate, 2.00 mmol of hexadecane, 0.20 mmol of Fe(acac)₃, 0.20 mmol of NaPF₆, 0.04 mmol of 1,2-dibromocyclohexane and 100 ml of dry toluene and the reaction mixture was heated to 80° C. in a stream of argon. In a glove box 0.04 mmol of the ruthenium compound (XIX or XX) and 0.04 (1:1) or 0.08 (1:2) mmol of a corresponding imidazolium salt (VI*HCl or VII*HCl) were dissolved together with 0.04 mmol (1:1) or 0.08 mmol (1:2) of NaO-t-Bu in 8 ml of dry toluene and introduced into the hot reaction solution in the 250 ml flask. The reaction solution was stirred at the desired reaction temperature for 3 hours in a stream of argon. After the reaction was complete, 0.1 ml of the reaction solution was dissolved in 1.5 ml of ethyl acetate and analysed by means of an HP 6890 gas chromatograph (GC). The specific reaction conditions and results are shown in Table 4.

TABLE 4
Examples 1 to 24

| | | | | | | | | RCM | |
|---|---|---|---|---|---|---|---|---|---|
| | Ru | | | Temperature | | Time | Conversion | product | Cycloisom. |
| Example | comp. | Lewis acid | Salt | (° C.) | Solvent | (h) | (%)^{[b]} | (%)^{[b]} | (%)^{[b]} |
| 1^{[a]} | XIV | — | — | 80 | Toluene | 1 | 65 | 33 | 32 |
| 2^{[a]} | XIV | — | NaPF₆ | 80 | Toluene | 1 | 76 | 76 | 0 |
| 3^{[a],[c]} | XIV | — | NaPF₆ | 80 | Toluene | 1 | 67 | 21 | 43 |
| 4^{[a],[d]} | XIV | — | NaPF₆ | 80 | Toluene | 1 | 72 | 63 | 8 |
| 5^{[a]} | XIV | AlCl₃ | — | 80 | Toluene | 1 | 34 | 34 | 0 |
| 6^{[a]} | XIV | AlCl₃ | NaPF₆ | 80 | Toluene | 1 | 94 | 94 | 0 |
| 7^{[a]} | XIV | PdCl₂(PPh₃)₂ | NaPF₆ | 80 | Toluene | 1 | 64 | 61 | 0 |
| 8^{[a]} | XIV | Fe(OAc)₂ | NaPF₆ | 80 | Toluene | 3 | >99 | 97 | 3 |
| 9^{[a]} | XIV | Fe(acac)₃ | — | 80 | Toluene | 1 | 48 | 46 | 0 |
| 10^{[a],[e]} | XIV | Fe(acac)₃ | NaPF₆ | 40 | Toluene | 3 | 12 | 10 | 1 |
| 11^{[a],[e]} | XIV | Fe(acac)₃ | NaPF₆ | 60 | Toluene | 3 | 43 | 40 | 1 |
| 12^{[a],[e]} | XIV | Fe(acac)₃ | NaPF₆ | 70 | Toluene | 3 | 78 | 78 | 0 |
| 13^{[a],[e]} | XIV | Fe(acac)₃ | NaPF₆ | 100 | Toluene | 3 | 76 | 76 | 0 |
| 14^{[a]} | XIV | Fe(acac)₃ | NaPF₆ | 140 | Xylene | 3 | 49 | 15 | 15 |
| 15^{[a],[e]} | XIX/VI | Fe(acac)₃ | NaPF₆ | 80 | Toluene | 3 | 46 | 43 | 2 |
| | (1:2) | | | | | | | | |
| 16^{[a],[c]} | XIX/VII | Fe(acac)₃ | NaPF₆ | 80 | Toluene | 3 | 24 | 23 | 1 |
| | (1:1) | | | | | | | | |
| 17^{[a],[e]} | XIX/VII | Fe(acac)₃ | NaPF₆ | 80 | Toluene | 3 | 56 | 52 | 3 |
| | (1:2) | | | | | | | | |
| 18^{[a],[e]} | XX/VII | Fe(acac)₃ | NaPF₆ | 80 | Toluene | 3 | 4 | 4 | 0 |
| | (1:2) | | | | | | | | |
| 19^{[a]} | XIII | Fe(acac)₃ | NaPF₆ | 80 | Toluene | 1 | 52 | 48 | 3 |
| 20^{[a]} | XVI | Fe(acac)₃ | NaPF₆ | 80 | Toluene | 1 | 24 | 22 | 0 |
| 21^{[a]} | XV | Fe(acac)₃ | NaPF₆ | 80 | Toluene | 1 | 22 | 14 | 3 |
| 22^{[a]} | XVII | Fe(acac)₃ | NaPF₆ | 80 | Toluene | 1 | 47 | 44 | 0 |
| 23^{[a]} | XVIII | Fe(acac)₃ | NaPF₆ | 80 | Toluene | 1 | 4 | 3 | 0 |
| 24^{[a]} | XII | Fe(acac)₃ | NaPF₆ | 80 | Toluene | 1 | 4 | 3 | 0 |

^{[a]}Reaction conditions: diethyl diallylmalonate (4 mmol), Ru compound (1 mol %), NaPF₆ (5 mol %) and Lewis acid (5 mol %), solvent (100 ml), stream of argon;
^{[b]}determined by GC using hexadecane as internal standard;
^{[c]}NaPF₆ (1 mol %);
^{[d]}NaPF₆ (10 mol %);
^{[e]}1,2-dibromocyclohexane (1 mol %) added.

### Examples 25 to 41

### Influence of Halogen Compounds

The model catalyst system consisting of XIV, NaPF₆ and Fe(acac)₃ was examined to determine its activity and selectivity as a function of the presence of selected halogen compounds (H1-H17).

### Examples 25 to 41

A 50 ml three-neck flask was charged with 1.00 mmol of diethyl diallylmalonate, 0.50 mmol of hexadecane, 0.05 mmol of Fe(acac)₃, 0.05 mmol of NaPF₆, 0.01 mmol of halogen compound (H1 to H17) and 21 ml of dry toluene and the reaction mixture was heated to 80° C. in a stream of argon. In a glove box, 0.01 mmol of XIV was dissolved in 4 ml of dry toluene and introduced into the hot reaction solution in the 50 ml flask. The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). The specific reaction conditions and results are shown in Table 5.

TABLE 5
Influence of halogen compounds

| | | Conver- | RCM | Yield of |
|---|---|---|---|---|
| Exam- | | sion | product | cycloisomer |
| ple^{[a]} | Halogen compound | (%) ^{[b]} | (%) ^{[b]} | (%) ^{[b]} |
| 25 | Benzyl chloride (H1) | 42 | 35 | 4 |
| 26 | Benzyl bromide (H2) | 41 | 38 | 1 |
| 27 | (1,2- | 50 | 42 | 4 |
| | Dibromoethyl)benzene(H3) | | | |
| 28 | Cyclohexyl iodide (H4) | 34 | 34 | 0 |
| 29 | Dibromomethane (H5) | 70 | 58 | 3 |
| 30 | 1,2-Dibromocyclohexane | 99 | 87 | 3 |
| | (H6) | | | |
| 31 | Cyclohexyl bromide (H7) | 71 | 68 | 3 |
| 32 | 1,2-Dibromoethane (H8) | 76 | 71 | 4 |
| 33 | 1,2-Dibromo-4,5- | 80 | 74 | 2 |
| | dimethylbenzene (H9) | | | |
| 34 | 1,2-Diiodobenzene (H10) | 86 | 84 | 2 |
| 35 | 1-Bromo-2-iodobenzene | 74 | 70 | 4 |
| | (H11) | | | |
| 36 | 2-Bromostyrene (H12) | 84 | 79 | 1 |
| 37 | (2-Bromoethyl)benzene | 74 | 71 | 2 |
| | (H13) | | | |
| 38 | (3-Bromopropyl)benzene | 72 | 70 | 2 |
| | (H14) | | | |
| 39 | Tetrabutylammonium | 82 | 80 | 2 |
| | bromide (H15) | | | |
| 40 | KBr (H16) | 60 | 50 | 2 |
| 41 | KI (H17) | 63 | 53 | 2 |

^{[a]}Reaction conditions: diethyl diallylmalonate (1 mmol), (p-cymene)Ru(Me₂IMes)Cl₂ (XIV) (1 mol %), NaPF₆ (5 mol %), Fe(acac)₃ (5 mol %), halogen compound (1 mol %), toluene (25 ml), stream of argon, 80° C., 3 h;
^{[b]} determined by GC using hexadecane as internal standard.

### Example 42

### Synthesis of diethyl 3-methylcyclohex-3-ene-1,1-dicarboxylate

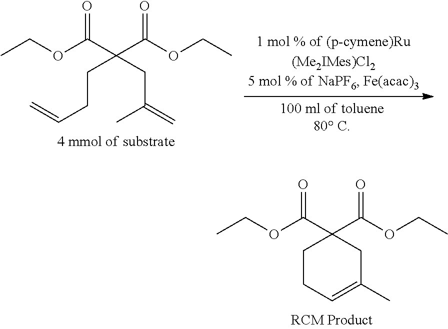 [SMILES: C=CCCC(CC(=C)C)(C(=O)OCC)C(=O)OCC ; CCOC(=O)C1(C(=O)OCC)CCC=C(C)C1]

A 250 ml three-neck flask was charged with 4.00 mmol of diethyl 2-(but-3-enyl)-2-(2-methylallyl)malonate, 2.00 mmol hexadecane, 0.2 mmol of Fe(acac)₃, 0.2 mmol of NaPF₆ and 100 ml of dry toluene and the reaction mixture was heated to 80° C. in a stream of argon. In a glove box, 0.04 mmol of XIV was dissolved in 8 ml of dry toluene and introduced into the hot reaction solution in the 250 ml flask. The reaction solution was stirred at 80° C. for 3 hours in a stream of argon. After the reaction was complete, 0.1 mmol of the reaction solution was dissolved in 1.5 ml of ethyl acetate and analysed by means of an HP 6890 gas chromatograph (GC). In the presence of XIV, NaPF₆ and Fe(acac)₃, a conversion of 50% and an RCM yield of 24% were achieved.

### Example 43

### Synthesis of 1-tosyl-2,5-dihydro-1H-pyrrole

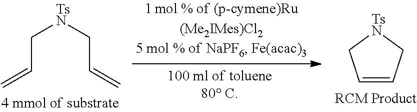 [SMILES: C=CCN(CC=C)S(=O)(=O)c1ccc(C)cc1 ; Cc1ccc(S(=O)(=O)N2CC=CC2)cc1]

A 250 ml three-neck flask was charged with 4.00 mmol of N,N-diallyl-4-methylbenzenesulphonamide, 2.00 mmol of hexadecane, 0.2 mmol of Fe(acac)₃, 0.2 mmol of NaPF₆ and 100 ml of dry toluene and the reaction mixture was heated to 80° C. in a stream of argon. In a glove box, 0.04 mmol of XIV was dissolved in 8 ml of dry toluene and introduced into the hot reaction solution in the 250 ml flask. The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). In the presence of XIV, NaPF₆ and Fe(acac)₃, a conversion of 14% and an RCM yield of 12% were achieved.

### Example 44

### Synthesis of 1-benzyloxycyclohex-3-ene

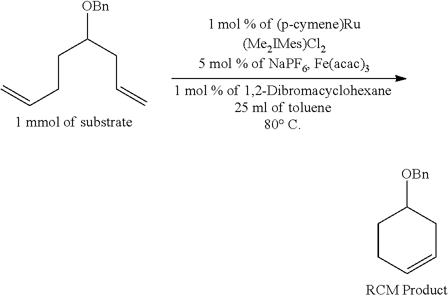 [SMILES: C=CCCC(CC=C)OCc1ccccc1 ; C1=CCC(OCc2ccccc2)CC1]

A 50 ml three-neck flask was charged with 1.00 mmol of 4-benzyloxy-octa-1,7-diene, 0.50 mmol of hexadecane, 0.05 mmol of Fe(acac)₃, 0.05 mmol of NaPF₆, 0.01 mmol of 1,2-dibromocyclohexane and 21 ml of dry toluene and the reaction mixture was heated to 80° C. in a stream of argon. In a glove box, 0.01 mmol of XIV was dissolved in 4 ml of dry toluene and introduced into the hot reaction solution in the 250 ml flask. The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). In the presence of XIV, NaPF₆, Fe(acac)₃ and 1,2-dibromocyclohexane, a conversion of 85% and an RCM yield of 41% were achieved.

### Example 45

### Synthesis of 1-benzyloxycyclopent-3-ene

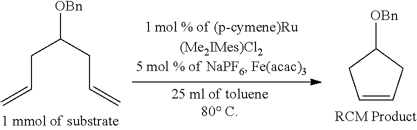

A 50 ml three-neck flask was charged with 1.00 mmol of 4-benzyloxyhepta-1,7-diene, 0.50 mmol of hexadecane, 0.05 mmol of Fe(acac)₃, 0.05 mmol of NaPF₆, 0.01 mmol of 1,2-dibromocyclohexane and 21 ml of dry toluene and the reaction mixture was heated to 80° C. in a stream of argon. In a glove box, 0.01 mmol of XIV was dissolved in 4 ml of dry toluene and introduced into the hot reaction solution in the 250 ml flask.

The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). In the presence of XIV, NaPF₆, Fe(acac)₃ and 1,2-dibromocyclohexane, a conversion of 90% and an RCM yield of 81% were achieved.

### Example 46

### Synthesis of 1-benzyloxy-1-methyl-cyclopent-3-ene

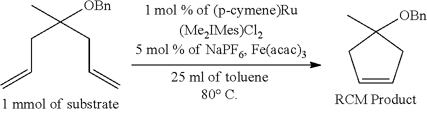 [SMILES: C=CCC(C)(CC=C)OCc1ccccc1 ; CC1(OCc2ccccc2)CC=CC1]

A 50 ml three-neck flask was charged with 1.00 mmol of 4-benzyloxy-4-methylhepta-1,7-diene, 0.50 mmol of hexadecane, 0.05 mmol of Fe(acac)₃, 0.05 mmol of NaPF₆ and 21 ml of dry toluene and the reaction mixture was heated to 80° C. in a stream of argon. In a glove box, 0.01 mmol of XIV was dissolved in 4 ml of dry toluene and introduced into the hot reaction solution in the 50 ml flask. The reaction solution was stirred at the desired reaction temperature for 3 hours in a stream of argon. After the reaction was complete, 0.1 ml of the reaction solution was dissolved in 1.5 ml of ethyl acetate and analysed by means of an HP 6890 gas chromatograph (GC). In the presence of XIV, NaPF₆ and Fe(acac)₃, a conversion of 95% and an RCM yield of 94% were achieved.

### Examples 47 and 48

### Synthesis of 2,5-dihydrobenzo[b]oxepin

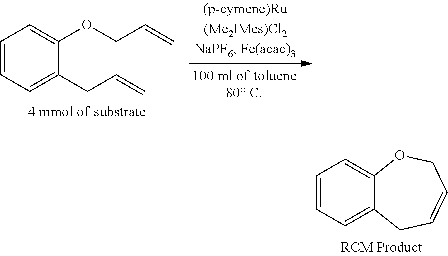

### Example 47

A 250 ml three-neck flask was charged with 4.00 mmol of 1-allyl-2-(allyloxy)benzene, 2.00 mmol of hexadecane, 0.2 mmol of Fe(acac)₃, 0.2 mmol of NaPF₆ and 100 ml of dry toluene and the reaction mixture was heated to 80° C. in a stream of argon. In a glove box, 0.04 mmol of XIV was dissolved in 8 ml of dry toluene and introduced into the hot reaction solution in the 250 ml flask. The reaction solution was stirred at the desired reaction temperature for 3 hours in a stream of argon. After the reaction was complete, 0.1 ml of the reaction solution was introduced in a countercurrent of argon into a GC vial, dissolved in 1.5 ml of ethyl acetate and analysed by means of an HP 6890 gas chromatograph (GC). In the presence of 1 mol % of XIV, NaPF₆ and Fe(acac)₃, a conversion of 94% and an RCM yield of 85% were achieved.

### Example 48

A 250 ml three-neck flask was charged with 4.00 mmol of 1-allyl-2-(allyloxy)benzene, 2.00 mmol of hexadecane, 0.02 mmol of Fe(acac)₃, 0.02 mmol of NaPF₆ and 100 ml of dry toluene and the reaction mixture was heated to 80° C. in a stream of argon. In a glove box, 0.004 mmol of XIV was dissolved in 8 ml of dry toluene and introduced into the hot reaction solution in the 250 ml flask. The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). In the presence of 0.01 mol % of XIV, NaPF₆ and Fe(acac)₃, a conversion of 94% and an RCM yield of 81% were achieved.

### Example 49

### Synthesis of (E)-1,2-diphenylethene (cross-metathesis

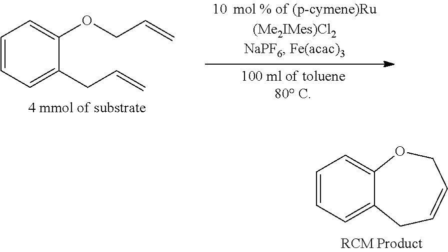

A 250 ml three-neck flask was charged with 4.00 mmol of styrene, 2.00 mmol of hexadecane, 2.0 mmol of Fe(acac)₃, 2.0 mmol of NaPF₆ and 80 ml of dry toluene and the reaction mixture was heated to 80° C. in a stream of argon. In a glove box, 0.4 mmol of XIV was dissolved in 20 ml of dry toluene and introduced into the hot reaction solution in the 250 ml flask. The reaction solution was stirred at 80° C. for 3 hours in a stream of argon. After the reaction was complete, 0.1 ml of the reaction solution was dissolved in 1.5 ml of ethyl acetate and analysed by means of an HP 6890 gas chromatograph (GC). In the presence of XIV, NaPF₆ and Fe(acac)₃, a conversion of 23% and a CM yield of 13% were achieved.

1. A process for preparing olefins by means of metathesis comprising the following steps
a. providing an olefin reaction mixture comprising at least one olefin,
b. adding a ruthenium compound of the general formula [RuX₂L¹ₓL²_{y}]_{z} (I), where X is an anionic ligand; L¹ is an uncharged π-bonding ligand; L² is an uncharged electron donor ligand; x=0 or 1; y=1, 2, or 3; z=1 or 2,
c. adding a Lewis acid or adding a Lewis acid and an anionic, noncoordinating salt,
d. reacting at temperatures in the range from 30° C. to 140° C.,
wherein no addition of an alkyne or alkynol is carried out.

2. The process according to claim 1, wherein the anionic ligands X are identical and are each chlorine and L² is selected from the group consisting of nitrogen bases, phosphanes, phosphinites, phosphonites, phosphites and arsanes.

3. The process according to claim 2, wherein L¹ is selected from the group consisting of benzene, toluene, xylene, cymene, trimethylbenzene, tetramethylbenzene, hexamethylbenzene, tetrahydronaphthalene and naphthalene, and L² is selected from the group consisting of N-heterocyclic carbenes and phosphanes.

4. The process according to claim 3, wherein L² is selected from the group consisting of P(phenyl)₃, P(cyclohexyl)₃ and N-heterocyclic carbenes of the formula VI, VII, VIII, IX, X and XI.

5. The process according to claim 1, wherein the ruthenium compound is selected from the group consisting of (a) compounds derived from formula (I) wherein x=1, y=1, z=1 (formula II), (b) compounds derived from formula (I) wherein x=0, y=2, z=1 (formula III) and (c) compounds derived from formula (I) wherein x=0, y=1, z=2 (formula IV)

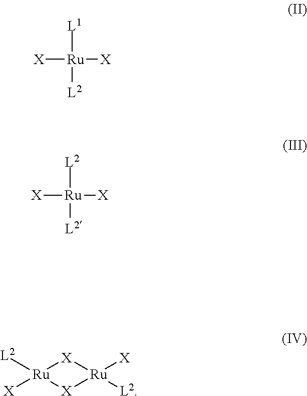

6. The process according to claim 5, wherein the ruthenium compound is a compound of the general formula RuX₂L¹L² (II), wherein the anionic ligands X are identical and are each chlorine and L² is selected from the group consisting of N-heterocyclic carbenes and phosphanes.

7. The process according to claim 6, wherein L¹ is selected from the group consisting of benzene, toluene, xylene, cymene, trimethylbenzene, tetramethylbenzene, hexamethylbenzene, tetrahydronaphthalene and naphthalene, and L² is selected from the group consisting of P(cyclohexyl)₃ and the N-heterocyclic carbenes of the formulae VI, VII, VIII, IX, X and XI.

8. The process according to claim 7, wherein the ruthenium compound is selected from the group consisting of compounds of the formulae A, B and C.

9. The process according to claim 5, wherein the ruthenium compound is a compound of the general formula RuX₂L²₂ (III), wherein the anionic ligands X are identical and are each chlorine and each of the ligands L² are selected independently from the group consisting of N-heterocyclic carbenes and phosphanes.

10. The process according to claim 9, wherein each of the ligands L² are selected independently from the group consisting of P(cyclohexyl)₃, P(phenyl)₃ and the N-heterocyclic carbenes of the formulae VI, VII, VIII, IX, X and XI.

11. The process according to claim 10, wherein one L² is selected from the group consisting of P(cyclohexyl)₃ and P(phenyl)₃ and the other L² is selected from the group consisting of the N-heterocyclic carbenes of the formulae VI, VII, VIII, IX, X and XI.

12. The process according to claim 5, wherein the ruthenium compound is a compound of the general formula [RuX₂L²]₂ (IV), where the anionic ligands X are identical and are each chlorine and each of the ligands L² are selected independently from the group consisting of N-heterocyclic carbenes and phosphanes.

13. The process according to claim 12, wherein the ligands L² are identical and are selected from the group consisting of P(cyclohexyl)₃ and the N-heterocyclic carbenes of the formulae VI, VII, VIII, IX, X and XI.

14. The process according to claim 1, wherein the anionic, noncoordinating salt is selected from the group consisting of a sodium, potassium, caesium, barium, calcium or magnesium salt of PF₆⁻, BF₄⁻, BH₄⁻, F₃CSO₃⁻, H₃CSO₃⁻, ClO₄⁻, SO₄²⁻, HSO₄⁻, NO₃⁻, PO₄³⁻, HPO₄²⁻, H₂PO₄⁻, CF₃COO⁻, B(C₆F₅)₄⁻, B[3,5-(CF₃)₂C₆H₃]₄⁻, RSO₃⁻ and R′COO⁻, wherein each of R and R′ are selected independently from the group consisting of (C₁-C₂₀)-alkyl and (C₆-C₁₄)-aryl.

15. The process according to claim 1, wherein the Lewis acid is selected from the group consisting of aluminium, boron, chromium, cobalt, iron, copper, magnesium, lanthanum, manganese, nickel, palladium and zinc salts of Cl⁻, Br⁻, I⁻, PF₆⁻, BF₄⁻, CF₃COO⁻, B(C₆F₅)₄⁻, B[3,5-(CF₃)₂C₆H₃]₄⁻, R^{x}COO⁻, (R^{y}COCHCOR^{z})⁻, wherein each of R^{x}, R^{y}, and R^{z} are selected independently from the group consisting of (C₁-C₂₀)-alkyl and (C₆-C₁₄)-aryl.

16. The process according to claim 1, wherein step c) further comprises adding a halogen compound.

17. The process according to claim 16, wherein the halogen compound is selected from the group consisting of potassium iodide, potassium bromide, tetrabutylammonium bromide, 1,2-dibromocyclohexane, 1,2-bromocyclohexane, 1,2-dibromoethane, 1,2-dibromo-4,5-dimethylbenzene, 1,2-diiodobenzene, 1-bromo-2-iodobenzene, 2-bromostyrene, (2-bromoethyl)benzene and (3-bromopropyl)benzene.

18. The process according to claim 1, wherein a ratio of the ruthenium compound to the at least one olefin is in the range from 1:10 to 1:1 000 000.

19. The process according to claim 1, wherein a ratio of the ruthenium compound to the anionic, noncoordinating salt is in the range from 1:1 to 1:10.

20. The process according to claim 1, wherein a ratio of the ruthenium compound to the Lewis acid is in the range from 1:1 to 1:10.

21. The process according to claim 16, wherein a ratio of the ruthenium compound to the halogen compound is in the range from 1:1 to 1:333.

22. Use of the process according to claim 1, wherein the process is used in metathesis reactions selected from the group consisting of cross-metathesis (CM), ring-closing metathesis (RCM), ring-opening metathesis (ROM), ring-opening metathesis polymerization (ROMP) and acyclic diene metathesis (ADMET).
